(19) United States
(12) Patent Application Publication
(10) Pub. No.: US 2015/0335242 A1
(43) Pub. Date: Nov. 26, 2015

(54) OPHTHALMIC APPARATUS AND CONTROL METHOD FOR THE SAME

(71) Applicant: CANON KABUSHIKI KAISHA, Tokyo (JP)

(72) Inventor: Yohei Saito, Kawasaki-shi (JP)

(21) Appl. No.: 14/714,476

(22) Filed: May 18, 2015

(30) Foreign Application Priority Data
May 22, 2014 (JP) 2014-106241

(51) Int. Cl. A61B 3/12; A61B 3/14

(52) U.S. Cl. CPC A61B 3/1216; A61B 3/14

(57) ABSTRACT

A fundus camera has an image pickup element adapted to receive reflection light from an anterior ocular segment of an examinee's eye and a light receiving optical system that guides the reflection light from the anterior ocular segment of the examinee's eye to the image pickup element. The fundus camera is provided with a focus evaluation value acquisition unit that acquires a focus evaluation value representing an in-focus state of the light receiving optical system for the anterior ocular segment of the examinee's eye on the basis of an output of the image pickup element for a specific part of the anterior ocular segment of the examinee's eye and an in-focus position determination unit that determines an in-focus position of the light receiving optical system based on the focus evaluation value. With these unit, auto-focusing can be performed when imaging the anterior ocular segment.

### BACKGROUND OF THE INVENTION

1. Field of the Invention

The present invention relates to an ophthalmic apparatus and a control method for an ophthalmic apparatus.

2. Description of the Related Art

In eye examinations, images of the anterior segment of an examinee's eye are picked up using an ophthalmic apparatus such as a fundus camera in some cases. In cases where a conventional fundus camera is used for this purpose, the examiner firstly adjusts the distance between the body of the fundus camera and the examinee's eye to bring the anterior segment of the eye in focus, and then captures an image(s). In doing so, the examiner determines the in-focus position by his/her own visual estimation, and a satisfactory image(s) are not picked up in some cases.

Japanese Patent Application Laid-Open 2012-050592 discloses an apparatus in which a focus lens is shifted to a predetermined position when the anterior segment of an eye is to be imaged to facilitate focusing operation by adjusting the distance between the apparatus body and an examinee's eye.

As described above, in conventional fundus cameras, in order to pick up a satisfactory image of the anterior segment of the examinee's eye, it is necessary to judge the in-focus state of the examinee's eye at a desired position by visual estimation and to determine the in-focus position of the optical system that brings the anterior segment of the eye in focus. Some known fundus cameras have an auto-focusing function for achieving focusing using a focus index, which indicates the in-focus state using light reflected from the ocular fundus to facilitate focusing operation. However, while the focus index using reflection from the ocular fundus enables detection of the in-focus position of the optical system for the ocular fundus, the in-focus position for the anterior segment of the eye cannot be detected. In the apparatus disclosed in Japanese Patent Application Laid-Open No. 2012-050592 also, it is difficult to bring the anterior segment in focus satisfactorily.

### SUMMARY OF THE INVENTION

The present invention has been made in view of the above circumstances, and an object of the present invention is to provide an ophthalmic apparatus that can achieve a satisfactory in-focus state for the anterior segment of an eye when imaging the anterior segment.

To achieve the above object, according to the present invention, there is provided a fundus camera comprising an image pickup element adapted to receive reflection light from an anterior ocular segment of an examinee's eye, a light receiving optical system that guides the reflection light from the anterior ocular segment of the examinee's eye to the image pickup element, a focus evaluation value acquisition unit that acquires a focus evaluation value representing an in-focus state of the light receiving optical system for the anterior ocular segment of the examinee's eye on the basis of an output of the image pickup element for a specific part of the anterior ocular segment of the examinee's eye, and an in-focus position determination unit that determines an in-focus position of the light receiving optical system based on the focus evaluation value.

The fundus camera according to the present invention can determine an in-focus position of the optical system automatically even when the anterior ocular segment is imaged.

Further features of the present invention will become apparent from the following description of exemplary embodiments with reference to the attached drawings.

### BRIEF DESCRIPTION OF THE DRAWINGS

FIG. 1 is a diagram schematically showing the general configuration of the fundus camera according to a first embodiment of the present invention.

FIG. 2 schematically shows the anterior ocular segment of the examinee's eye to illustrate a method of calculating a focus evaluation value in the first embodiment of the present invention.

FIG. 3 shows the change of the contrast value in relation to the position of the focus lens in the first embodiment of the present invention.

FIG. 4 is a flow chart showing a sequence of anterior ocular imaging in the fundus camera according to the first embodiment of the present invention.

FIG. 5 schematically shows the anterior ocular segment of the examinee's eye to illustrate a method of calculating a focus evaluation value in a second embodiment of the present invention.

FIG. 6 shows the change of the contrast value in relation to the position of a movable base in the second embodiment of the present invention.

FIG. 7 is a flow chart showing a sequence of anterior ocular imaging in the fundus camera according to the second embodiment of the present invention.

### DESCRIPTION OF THE EMBODIMENTS

Preferred embodiments of the present invention will now be described in detail in accordance with the accompanying drawings. The embodiments described in the following is not intended to limit the present invention as defined in the claims, and combinations of the features of the present invention described with the embodiment are not necessarily essential to the present invention.

### First Embodiment

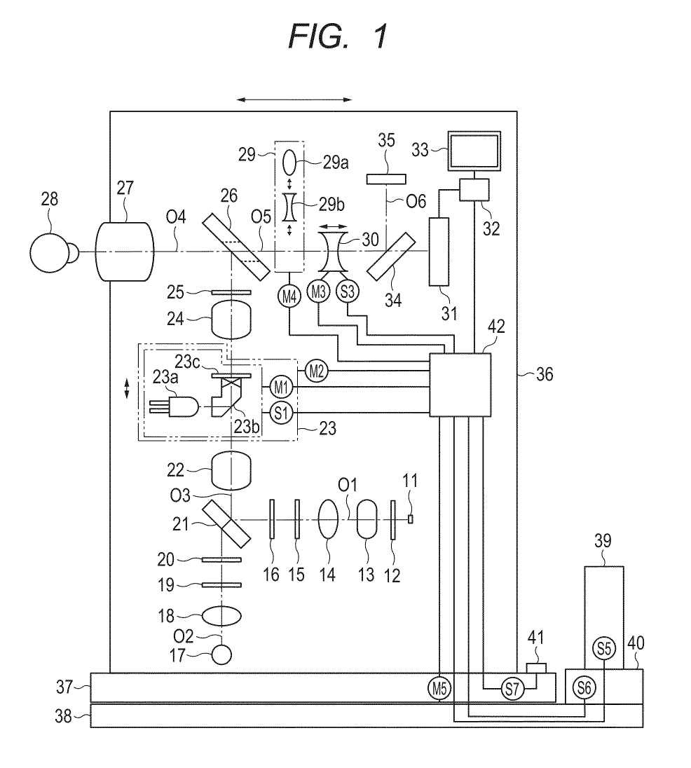

A fundus camera as an example of an ophthalmic apparatus to which the present invention is applied will be described as a first embodiment with reference to FIGS. 1 to 4. FIG. 1 is a diagram schematically showing the general configuration of the fundus camera according to the first embodiment of the present invention.

The optical system of this fundus camera is roughly divided into an imaging light source section O1, an observation light source section O2, an illumination optical system O3, an imaging/illumination optical system O4, an imaging optical system O5, and an internal fixation lamp section O6. Light beams emitted from the imaging light source section O1 or the observation light source section 2 pass through the illumination optical system O3, the imaging/illumination optical system O4 and illuminate the ocular fundus of an eye of an examinee. An image of the ocular fundus of the examinee is formed on an image pickup element through the imaging/illumination optical system O4 and the imaging optical system O5.

The imaging light source section O1 includes components described below and provides ring illumination with white light. The imaging light source section O1 includes a light intensity detection unit 11, a mirror 12, an imaging light source 13, an imaging condenser lens 14, an imaging ring slit 15, and an imaging crystalline lens baffle 16. The light intensity detection unit 11 is a photoelectric conversion sensor such as SPC or PD. The mirror 12 transmits light in a region near the optical axis and reflects light outside the region near the optical axis. The mirror 12 may be composed of a glass plate having a deposited silver or aluminum film, or an aluminum plate. The imaging light source 13 includes a glass tube in which xenon gas is sealed and emits light with voltage application. The imaging light source 13 can provide white light having an intensity high enough to enable imaging of the ocular fundus. Alternatively, the imaging light source 13 may be composed of annularly arranged LED array, using large light-quantity LEDs developed in recent years. The imaging condenser lens 14 is an ordinary spherical lens. The imaging ring slit 15 is a flat plate having an annular opening. The imaging crystalline lens baffle 16 is also a flat plate having an annular opening.

The beams emitted from the imaging light source 13 are partly directed toward the ocular fundus. Moreover, beams emitted to the opposite direction are reflected by the mirror 12 and directed toward the ocular fundus. Therefore, the quantity of light emitted from the imaging light source 13 may be smaller than in the case where mirror 12 is not provided. The reflecting surface of the mirror 12 is a flat surface, which does not create unevenness in light distribution and is not limited in terms of its distance from the imaging light source 13. The light beams are condensed by the condenser lens 14 toward the ocular fundus and then shaped by the imaging ring slit 15 in such a way as to have an annular shape when the beams pass through the anterior ocular segment of the eye. The imaging crystalline lens baffle 16 restricts beams cast onto crystalline lens of the examinee's eye and prevents an image of the ocular fundus from being interfered with unnecessary reflection light from the crystalline lens of the examinee's eye.

The observation light source section O2 includes components described below and provides ring illumination with infrared light. The observation light source section O2 includes an observation light source 17, an observation condenser lens 18, an observation ring slit 19, and an observation crystalline lens baffle 20. The observation light source 17 is a light source capable of continuously emitting light, such as a halogen lamp or LED, which is adapted to emit infrared light by its characteristic or using an optical filter. The observation condenser lens 18 is an ordinary spherical lens. The observation ring slit is a flat plate having an annular opening. The observation crystalline lens baffle 20 is also a flat plate having an annular opening. The observation light source section O2 differs from the imaging light source section O1 only in the type of the light source. Light beams are condensed by the observation condenser lens 18, the shape of beams at the anterior ocular segment is shaped by the observation ring slit 19, and the observation crystalline lens baffle 20 prevents an image of the ocular fundus from being interfered with light reflected from the crystalline lens of the examinee's eye.

The illumination optical system O3 relays beams produced by the imaging light source section O1 and the observation light source section O2 and produces an index image used for focusing of an image of the ocular fundus. The illumination optical system O3 includes a dichroic mirror 21, which transmits infrared light and reflects visible light. Visible light beams produced by the imaging light source section O1 are reflected by the dichroic mirror 21, and infrared light beams produced by the observation light source section O2 are transmitted through the dichroic mirror 21, so that both beams are delivered to the illumination optical system O3. Ring illumination is focused onto the examinee's eye by a first illumination relay lens 22 and a second illumination relay lens 24.

The apparatus has a split unit 23, which includes a focus index light source 23a, a prism 23b, a focus index mask 23c, a shift mechanism, and an advancing-retracting mechanism. The focus index light source 23a is adapted to project a focus index. The prism 23b is adapted to split the light source. The focus index mask 23c defines the outer shape of the focus index. The shift mechanism is adapted to insert these components into the illumination optical system O3 when the observation is performed and to shift them along the direction indicated by the double-sided arrow in FIG. 1, thereby shifting the focus index along the direction of the optical axis. The advancing-retracting mechanism retracts these component away from the illumination optical system O3, when imaging is performed.

The split unit 23 is shifted by a split unit shift drive motor M1, whereby the focus index is brought into focus. The position at which the split unit 23 is stopped is detected by a split position sensor S1. The spilt unit 23 is inserted into/retracted from the illumination optical system O3 by a split unit advancing/retracting motor M2. When the ocular fundus is observed, the split unit 23 is inserted into the illumination optical system O3 by the split unit advancing/retracting motor M2, so that the focus index is projected into the observed image. When imaging is performed, the split unit 23 is retracted from the illumination optical system O3 by the split unit advancing/retracting motor M2, so that the focus index is prevented from appearing in the captured image. The apparatus also has a cornea baffle 25, which prevents unwanted reflection light from the cornea of the examinee's eye from being cast into the image of the ocular fundus.

The imaging observation optical system O4 casts illumination light beams onto the ocular fundus of the examinee's eye 28 and forms an image of the ocular fundus of the examinee's eye. The imaging observation optical system O4 has an apertured mirror 26 having an aperture at the center and an outer mirror portion around the aperture. Light beams coming from the illumination optical system O3 are reflected by the mirror portion of the apertured mirror 26 to illuminate the ocular fundus of the examinee's eye through the objective lens 27. Beams for forming an image of the ocular fundus of the examinee's eye thus illuminated return through the objective lens 27, pass through the center aperture of the apertured mirror 26, and enter the imaging optical system O5.

The imaging optical system O5 forms an image of the ocular fundus of the examinee's eye on an image pickup element 31 with its focus being adjusted. The imaging optical system O5 includes a focus lens 30 for focus adjustment of imaging beams passing through the center aperture of the apertured mirror 26. Specifically, the focus lens 30 shifts in the direction of the double-sided arrows in FIG. 1 to adjust the focus. A focus lens drive motor M3 is provided to drive the focus lens 30 for focus adjustment. The position at which the focus lens 30 is stopped is detected by a focus lens position sensor S3.

The imaging optical system O5 also includes a diopter correction lens 29. The diopter correction lens includes a convex lens and a concave lens, which can be set at a position on the optical axis and can be removed therefrom. The diopter correction lens 29 is used to bring the ocular fundus of the examinee's eye in focus, in cases where the examinee's eye is near-sighted or far-sighted so highly that it is difficult to attain focus adjustment satisfactorily by the focus lens 30. The focus lens 30 constitutes the focusing member as defined in the present invention. The focus lens 30 may be replaced by various optical components having the same function. A motor M4 for advancing and retracting the diopter correction lens is provided to insert a negative diopter correction lens 29b, in the case where the examinee's eye is highly near-sighted, and a positive diopter correction lens 29a, in the case where the examinee's eye is highly far-sighted.

The image pickup element 31 converts imaging light into an electrical signal by photoelectric conversion. The electrical signal generated by the image pickup element 31 is AD-converted into digital data by an image processing unit 32. An image is displayed on a monitor 33 during infrared observation. The digital data is recorded in a recording medium not shown in the drawings after the imaging.

The imaging optical system O5 has a half mirror 34, which splits out an optical path for the internal fixation lamp section O6 from the imaging optical system O5. An internal fixation lamp unit 35 is opposed to this optical path. The internal fixation lamp unit 35 has a plurality of LEDs. An LED located at the position corresponding to a fixation target selected by the examiner is turned on. While the examinee gazes the LED thus turned on, the examiner can capture an ocular fundus image in the desired orientation.

The above-described optical systems are fixedly mounted on a casing 36. The casing 36 is fixed to a movable base 37, which can be shifted relative to the fixed base 38 along the direction of the optical axis by a main body driving motor M5. The movable base 37 and the arrangement for shifting it along the optical axis of the image pickup element 31 constitute the main body driving unit as defined in the present invention. The apparatus also has a fixed base 38. When the examiner operates a main body operation member 39 provided on the fixed base 38, the stop position or operated position of the main body operation member 39 is detected by a main body operation sensor S5. The main body operation sensor S5 outputs a signal representing the detected position to a system control unit 42. The system control unit 42 drives the main body drive motor M5 by an amount represented by the signal from the main body operation sensor S5 or other control signal.

Similarly, when the examiner operates a focus operation member 40 provided on the fixed base, the stop position or operated position of the focus operation member 40 is detected by a focus operation member position sensor S6. The focus operation member position sensor S6 outputs a signal representing the detected position to the system control unit 42. The system control unit 42 drives the focus lens drive motor M3 by an amount represented by the signal from the focus operation member position sensor S6 or other control signal.

The movable base 37 is provided with an anterior ocular imaging mode switch 41. When the examiner operates the anterior ocular imaging mode switch 41, the operation is detected by an anterior ocular imaging mode sensor S7, and a signal representing the operation is output to the system control unit 42.

In this fundus camera, signals from all the above-described sensors are input to the system control unit 42. The system control unit 42 controls all of the above-described motors.

In connection with the above description, the image pickup element 31 in this embodiment constitutes the image pickup element that receives light reflected by the anterior ocular segment of the examinee's eye as defined in the present invention, and the optical system of the fundus camera constitutes the light receiving optical system that delivers light reflected from the anterior ocular segment of the examinee's eye to the image pickup element as defined in the present invention.

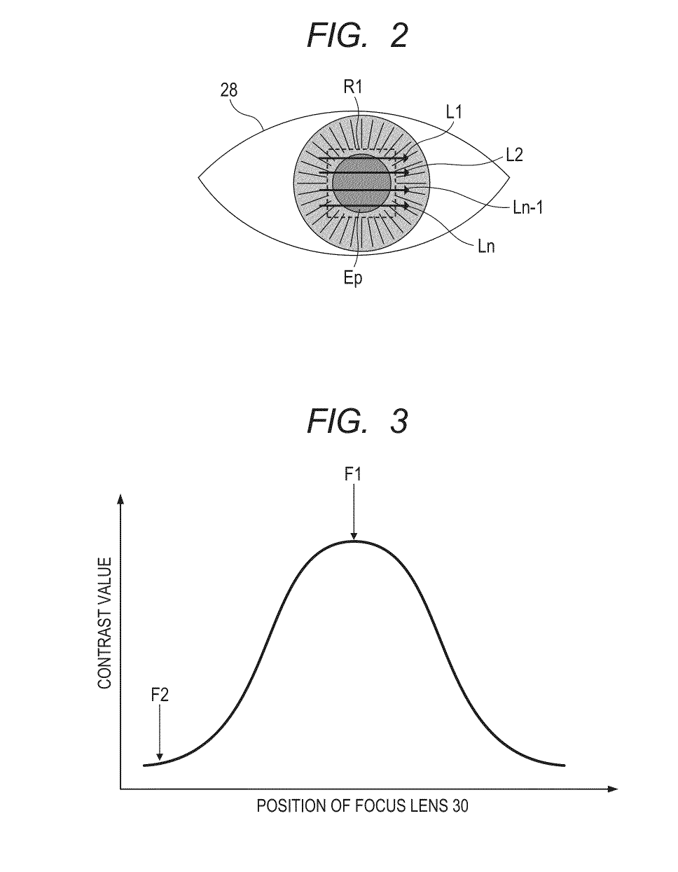

FIG. 2 schematically shows the anterior ocular segment of the examinee's eye 28 to illustrate a method of calculating a focus evaluation value in the first embodiment of the present invention. The focus evaluation value in the first embodiment is calculated based on a contrast value in a focus evaluation area R1 indicated in the picture of the anterior ocular segment in FIG. 2. The focus evaluation area R1 in this embodiment includes a specific part of the examinee's eye and an area around that part. In the first embodiment, the specific part is the pupil edge.

The focus evaluation area R1 is a square area of a size of n×n pixels having a center located at the center of the pupil Ep of the examinee's eye. The focus evaluation area R1 covers the entirety of the pupil. In FIG. 2, scanning lines L1 to Ln for evaluating the contrast value of the image are also shown. In this embodiment, all of the n pixels arranged along the vertical direction in FIG. 2 are scanned, and hence the number of scanning lines is n. However, the number of scanning lines may be changed, if necessary, in order to reduce the time taken to calculate the contrast value.

The size of the focus evaluation area R1 is determined in such a way as to cover the entirety of the pupil, and this size may be changed appropriately in accordance with the degree of contraction of the examinee's pupil. For example, the image processing unit 32 determines the focus evaluation area R1 on the basis of the size of the pupil. In order to use the edge of the pupil in calculating the focus evaluation value, the image processing unit 32 may set a focus evaluation area R1 having a size equal to the size of the pupil plus a predetermined value. If a focus evaluation area R1 covering the entirety of the pupil is set when the size (e.g. diameter) of the pupil is equal to or larger than a predetermined threshold, it takes a longer time to calculate the focus evaluation value than the time taken to calculate the focus evaluation value for the pupil having a size smaller than the predetermined threshold. Therefore, when the size of the pupil is equal to or larger than the predetermined threshold, the size of the focus evaluation area R1 may be set in such a way as to cover the half of the pupil rather than the entirety of the pupil. The portion of the pupil to be covered is not limited to half but it may be quarter or other proportions. In other words, the size of the focus evaluation area R1 may be changed based on the size of the pupil. Thus, when a specific part is selected by an area selection unit, it is preferred that the position and/or the size of the area in which a focus evaluation value (described later) is acquired be changed based on the size of the specific part.

In the above-described case, the size of the focus evaluation area R1 is changed based on whether or not the size of the pupil is equal to or larger than a threshold. However, the method employed is not limited to this. The size of the focus evaluation area R1 may be varied in multiple steps using a plurality of thresholds. Alternatively, the size of the focus evaluation area R1 may be decreased as the size of the pupil increases. The size of the pupil can be extracted from the anterior ocular image captured by the image pickup element 31.

The contrast mentioned in this context is a difference in brightness between adjacent pixels. The scanning lines are lines along which adjacent brightness values are calculated in order. The scanning lines extend in the horizontal direction in the image and arranged at regular intervals equal to one pixel size. The brightness value of each pixel is extracted along the scanning lines. The contrast value is defined to be the largest difference (absolute value) between adjacent brightness values in one scanning line.

The graph in FIG. 3 shows the change of the contrast value in relation to the position of the focus lens 30 shifted by the focus lens drive motor M3. In this illustrative case, the difference between the pupil portion and both ends of the edge of the portion other than the pupil is the dominant contrast component.

As shown in FIG. 3, since an in-focus image is sharp, the contrast value is largest at the in-focus position F1, and the contrast value is small at a greatly-out-of-focus position F2. Therefore, a satisfactory in-focus state can be achieved by shifting the focus lens 30 to a position at which the contrast value is maximized.

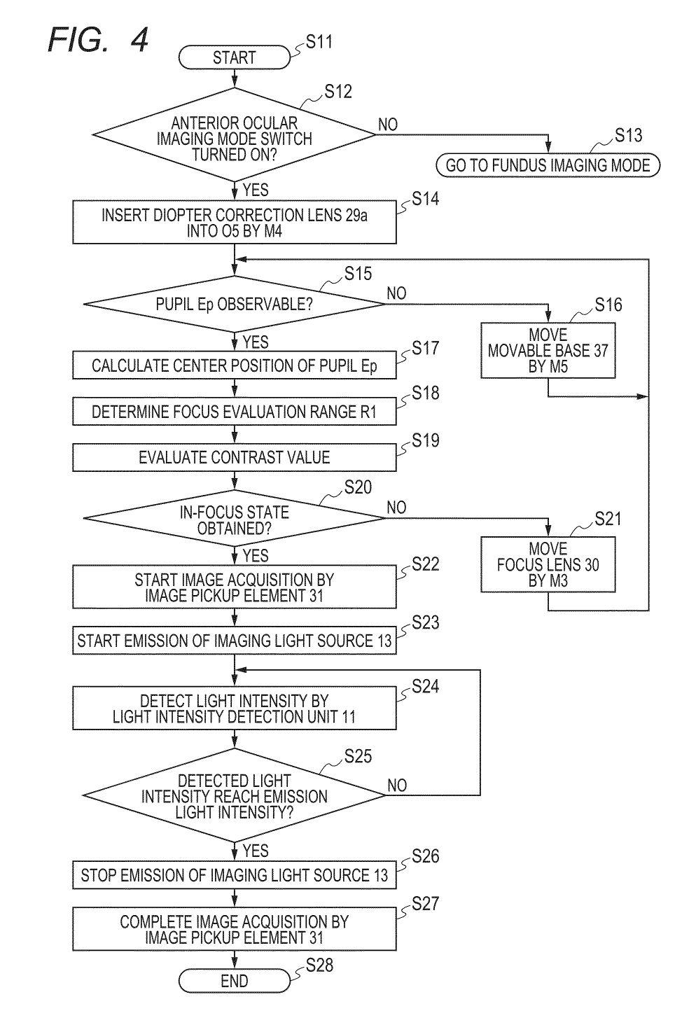

FIG. 4 is a flow chart showing a sequence of anterior ocular imaging in the fundus camera according to the first embodiment. In the following the anterior ocular imaging sequence in the fundus camera according to the first embodiment will be described. The anterior ocular imaging sequence is included in the system control unit 42 and executed by module areas corresponding to various means described later.

In step S11, imaging is started.

In step S12, it is determined whether or not an anterior ocular imaging mode switch 41 is on.

If it is determined that the anterior ocular imaging mode switch is on, the flow advances to step S14. If the anterior ocular imaging mode switch is not on, the flow advances to step S13.

In step S13, the mode is shifted to fundus imaging mode. (The fundus imaging mode is carried out according to ordinary fundus imaging procedure, which will not be described here.)

In step S14, the positive diopter correction lens 29a is inserted onto the imaging optical system O5 by the diopter correction lens advancing/retracting drive motor M4.

In step S15, it is determined whether or not the pupil can be observed by an output signal of the image pickup element 31.

If it is determined that the pupil can be observed, the flow advances to step S17. If it is determined that the pupil cannot be observed, the flow advances to step S16.

In step S16, the movable base 37 is driven by the main body drive motor M5.

In step S17, the position of the center of the pupil Ep is calculated from the output signal of the image pickup element 31. Specifically, the pupil is extracted from an image of the anterior ocular segment captured by the image pickup element 31, and the position of the center of the pupil is calculated. The extraction of the pupil and the calculation of the position of the pupil center can be carried out by various known methods. In this embodiment, they are carried out by computation based on the output signal of the image pickup element 31 (i.e. an image of the anterior ocular segment) acquired in step S15. Therefore, the center of the pupil is detected based on brightness information over the entire area of the image pickup element.

In step S18, a focus evaluation value calculation area R1 is determined based on the center of the pupil Ep.

In step S19, a contrast value in the focus evaluation value calculation area R1 is calculated.

In step S20, a determination as to in-focus state is made by determining whether the contrast value is largest or not.

If the contrast value is largest, it can be concluded that an in-focus state is achieved, and the flow advances to step S22. If the contrast value is not largest, the flow advances to step S21 to search for the largest value.

In step S21, the focus lens 30 is driven by the focus lens drive motor M3. After driving the focus lens 30, the steps from S15 to S20 are executed again. These steps are repeatedly executed until the largest contrast value or the in-focus state is attained.

In step S22, image acquisition by the image pickup element 31 is started, because the in-focus state is established when this step is executed.

In step S23, emission of light from the imaging light source 13 is started.

In step S24, the light intensity is detected by a light intensity detection unit 11.

In step S25, it is determined whether the detected light intensity reaches the emission light intensity.

If it is determined that the detected light intensity reaches the emission light intensity, the flow advances to step S26. If it is determined that the detected light intensity does not reach the emission light intensity, the flow advances to step S24, and the steps S24 and S25 are executed repeatedly until it is determined that the emission light intensity is reached.

When the emission light intensity is reached, light emission from the imaging light source 13 is stopped in step S26.

In step S27, image acquisition by the image pickup element 31 is ended.

In step S28, imaging is ended.

In the fundus camera described above, focusing can be performed automatically by driving the focus lens even during imaging of the anterior ocular segment. Moreover, since the contrast of the pupil edge is dominant in determining the in-focus state, imaging can be performed in a state in which the portion around the pupil is particularly in a satisfactory in-focus state.

The calculation of the contrast value performed in the above-described step S19 is executed by a module area in the system control unit 42 that functions as a focus evaluation value acquisition unit. The focus evaluation value acquisition unit acquires a focus evaluation value representing the in-focus state of the light receiving optical system for the anterior ocular segment, based on an output of the image pickup element 31 for a specific portion of the anterior ocular segment. In this illustrative embodiment, the contrast value is calculated. However, a value calculated and stored in a memory or the like may be retrieved as the contrast value. Therefore, in the present invention, the contrast value is defined to be an acquired value. The determination as to the in-focus state in step S20 is performed based on the focus evaluation value by a module area functioning as an in-focus position determination unit that determines an in-focus position in the light receiving optical system.

When the imaging operation is shifted to imaging of the ocular fundus, focus control is performed by a method other than the focus control method employed in imaging of the anterior ocular segment (contrast focus). In imaging of the ocular fundus, for example, a known focus control method using a split index is used. Autofocusing in imaging of the ocular fundus is more difficult than that in imaging of the anterior ocular. Therefore, changing the focus control method between the imaging of the anterior ocular and imaging of the ocular fundus is effecting in increasing the focus control speed.

### Second Embodiment

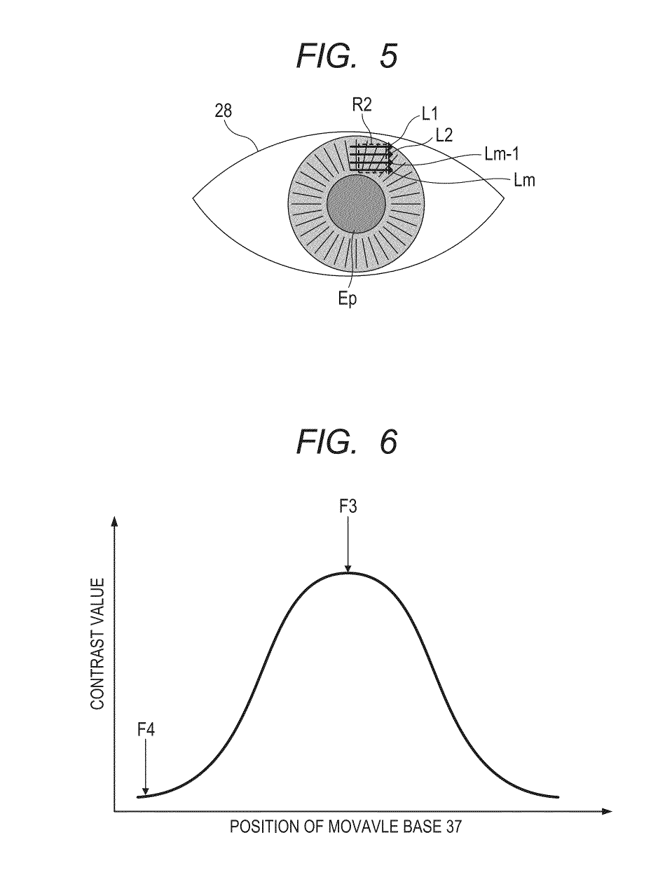

A second embodiment of the fundus camera employing the present invention will be described with reference to FIGS. 5 to 7. In the second embodiment, the examiner can flexibly select a contrast evaluation area. In the following, an illustrative case where an iris portion is used will be described.

The construction of the fundus camera in the second embodiment of the present invention is the same as that in the first embodiment.

FIG. 5 schematically shows the anterior ocular segment of the examinee's eye 28 to illustrate a method of calculating a focus evaluation value in the second embodiment of the present invention. The focus evaluation value in the second embodiment is calculated based on a contrast value in an area R2 indicated in the picture of the anterior ocular segment in FIG. 5. The area R2 is a square area of a size of m×m pixels having a center offset from the center of the pupil Ep of the examinee's eye. The area R2 is small enough to be within the area of the iris. In this case, an iris pattern is used as a specific part. The iris has a larger variety of patterns as compared to the white part and the pupil, and therefore it is advantageous for calculation of a contrast value.

The position of the center of the area R2 can be changed to a desired position around which the examiner wishes to measure the contrast specifically, using an area selection operation part not shown in the drawings. The operation member used in this operation is arranged, for example, adjacent to the anterior ocular imaging mode switch 41. This operation part function as an area selection unit used to select an area as a specific part. Although the apparatus according to the above-described first embodiment does not have such an area selection unit, the apparatus according to the first embodiment may also be equipped with such a unit.

In FIG. 5, scanning lines L1 to Ln for evaluating the contrast value of the image are also shown. The definition of the contrast value is the same as that in the first embodiment.

The graph in FIG. 6 shows the change of the contrast value in relation to the position of the movable base 37 shifted by the main body drive motor M5. In this illustrative case, the brightness difference in the iris patterns in the iris is the dominant contrast component.

As with in the first embodiment, since an in-focus image is sharp, the contrast value is largest at the in-focus position F3, and the contrast value is small at a greatly-out-of-focus position F4.

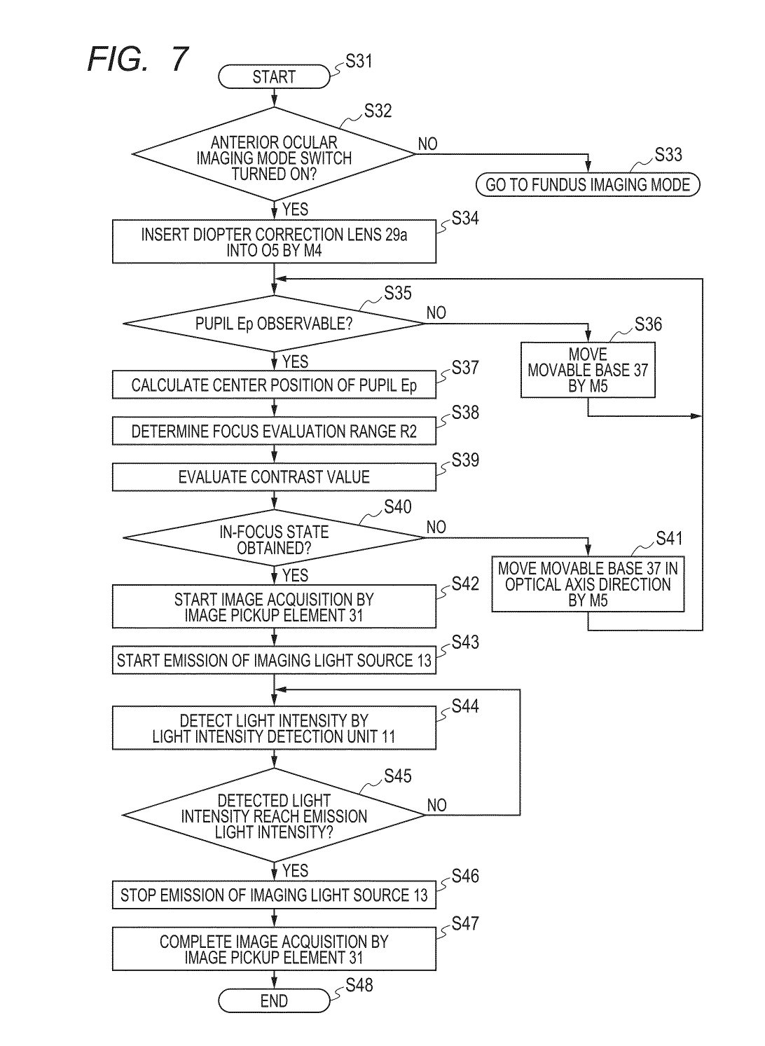

FIG. 7 is a flow chart showing a sequence of anterior ocular imaging in the fundus camera according to the second embodiment. In the following the anterior ocular imaging sequence in the fundus camera according to the second embodiment will be described.

In step S31, imaging is started.

In step S32, it is determined whether or not an anterior ocular imaging mode switch 41 is on.

If it is determined that the anterior ocular imaging mode switch is on, the flow advances to step S34. If the anterior ocular imaging mode switch is not on, the flow advances to step S33.

In step S33, the mode is shifted to fundus imaging mode. (The fundus imaging mode is carried out according to ordinary fundus imaging procedure, which will not be described here.)

In step S34, the positive diopter correction lens 29a is inserted onto the imaging optical axis O5 by the diopter correction lens advancing/retracting drive motor M4.

In step S35, it is determined whether or not the pupil can be observed by an output signal of the image pickup element 31.

If it is determined that the pupil can be observed, the flow advances to step S37. If it is determined that the pupil cannot be observed, the flow advances to step S36.

In step S36, the movable base 37 is driven by the main body drive motor M5.

In step S37, the position of the center of the pupil is calculated from the output signal of the image pickup element 31.

In step S38, a focus evaluation value calculation area R2 at a predetermined distance from the center of the pupil is determined. The predetermined distance used in step S38 may be either a fixed value or a variable value. Since the area of the iris changes depending on the degree of contraction of the pupil, the image processing unit 32 may determine the focus evaluation value calculation area R2 on the basis of the size of the pupil. When the size of the pupil is equal to or larger than a predetermined threshold, the area of the iris is located farther from the center of the pupil and the size of the iris area is smaller than when the size of the pupil is smaller than the predetermined threshold. Therefore, if the predetermined distance used in step S38 is varied based on the size of the pupil, it is possible to reduce the influence of pupil contraction and to set a focus evaluation value calculation area R2 reliably in the iris area. For example, as the size of the pupil increases, the predetermined distance used in step S38 may be increased. Moreover, as the size of the pupil increases, the area of the iris decreases. Therefore, as the size of the pupil increases, the size of the focus evaluation value calculation area R2 may be decreased. The predetermined distance used in step S38 may be changed (or increased) when the size of the pupil is equal to or larger than the predetermined threshold. Alternatively, the predetermined distance used in step S38 may be changed in multiple steps using a plurality of thresholds. Moreover, when the size of the pupil is equal to or larger than a predetermined threshold, the size of the focus evaluation value calculation area R2 may be changed (or decreased). The size of the focus evaluation value calculation area R2 may be changed in multiple steps using a plurality of thresholds.

The direction in which the focus evaluation value calculation area R2 is spaced from the center of the pupil by the predetermined distance may be either selected arbitrarily or predetermined. In the case where the direction is predetermined, the focus evaluation value calculation area R2 may be set, for example, at position spaced from the center of the pupil by the predetermined distance in the direction toward the ear, nose or lower eyelid. This can reduce the possibility that eyelashes appear in the image in the focus evaluation value calculation area R2.

In this process, the edge of the pupil may be extracted, and the focus evaluation value calculation area R2 may be determined in relation to the position of the edge.

In step S39, a contrast value in the focus evaluation value calculation area R2 is calculated.

In step S40, it is determined whether the contrast value is largest or not.

If the contrast value is largest, it can be concluded that an in-focus state is achieved, and the flow advances to step S42. If the contrast value is not largest, the flow advances to step S41 to search for the position of the movable base 37 that maximizes the contrast value.

In S41, the movable base 37 is driven by the main body drive motor M5. After driving the movable base 37, the steps from S35 to S40 are executed again. These steps are repeatedly executed until the largest contrast value or the in-focus state is attained.

In step S42, image acquisition by the image pickup element 31 is started, because the in-focus state is established when this step is executed.

In step S43, emission of light from the imaging light source 13 is started.

In step S44, the light intensity is detected by a light intensity detection unit 11.

In step S45, it is determined whether the detected light intensity reaches an emission light intensity.

If it is determined that the detected light intensity reaches the emission light intensity, the flow advances to step S46. If it is determined that the detected light intensity does not reach the emission light intensity, the flow advances to step S44, and the steps S44 and S45 are executed repeatedly until it is determined that the emission light intensity is reached.

When the emission light intensity is reached, light emission from the imaging light source 13 is stopped in step S46.

In step S47, image acquisition by the image pickup element 31 is ended.

In step S48, imaging is ended.

In the fundus camera described above, focusing can be performed automatically without need to drive the focus lens even during imaging of the anterior ocular segment. Moreover, since the contrast in iris patterns is dominant in determining the in-focus state, imaging can be performed in a state in which the portion around the iris is particularly in a satisfactory in-focus state. By changing the focus evaluation value calculation area, an image having good contrast at a desired location can be acquired. Moreover, since the in-focus determination area is selectable, an image having good image quality in a desired area can be acquired easily.

### Other Embodiments

The features of the above-described first and second embodiments may be employed in combination. For example, when the point at which the contrast value is maximized cannot be detected by the method according to one of the above-described embodiments, the other method may be employed. In cases where the size of the pupil is large and the area of the iris is small, it may be difficult to detect the point that maximizes the contrast value. Therefore, if the size of the pupil is equal to or larger than a threshold, the method according to the first embodiment may be employed rather than the method according to the second embodiment. In cases where the size of the pupil is large, the size of the area R1 is large, and focus control may take long time. Therefore, if the size of the pupil is equal to or larger than a threshold, the method according to the second embodiment may be employed rather than the method according to the first embodiment.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-106241, filed May 22, 2014 which is hereby incorporated by reference herein in its entirety.

What is claimed is:

1. An ophthalmic apparatus comprising:
an image pickup element adapted to receive reflection light from an anterior ocular segment of an examinee's eye;
a light receiving optical system that guides the reflection light from the anterior ocular segment of the examinee's eye to the image pickup element;
a focus evaluation value acquisition unit that acquires a focus evaluation value representing an in-focus state of the light receiving optical system for the anterior ocular segment of the examinee's eye on the basis of an output of the image pickup element for a specific part of the anterior ocular segment of the examinee's eye; and
an in-focus position determination unit that determines an in-focus position of the light receiving optical system based on the focus evaluation value.

2. An ophthalmic apparatus according to claim 1, wherein the specific part is a pupil edge.

3. An ophthalmic apparatus according to claim 1, wherein the specific part is an iris pattern.

4. An ophthalmic apparatus according to claim 1, further comprising an area selection unit that selects an area as the specific part.

5. An ophthalmic apparatus according to claim 4, wherein the area selection unit changes at least one of the position and the size of an area in which the focus evaluation value is acquired, dependently on the size of the specific part.

6. An ophthalmic apparatus according to claim 1, wherein the light receiving optical system includes a focusing member which can be shifted along the direction of the optical axis of the image pickup element, and the in-focus position determination unit changes the position of the focusing member on the optical axis relative to the examinee's eye.

7. An ophthalmic apparatus according to claim 1, further comprising a main body drive unit that drives the light receiving optical system and the image pickup element along the direction of the optical axis of the image pickup element, wherein the in-focus position determination unit changes the position of the light receiving optical system on the optical axis by means of the main body drive unit.

8. A recording medium in which a program that causes a computer to function as the units of the ophthalmic apparatus according to claim 1 is stored.

9. A control method for an ophthalmic apparatus employed in a fundus camera having an image pickup element adapted to receive reflection light from an anterior ocular segment of an examinee's eye and a light receiving optical system that guides the reflection light from the anterior ocular segment of the examinee's eye to the image pickup element, comprising:
a focus evaluation value acquisition step of acquiring a focus evaluation value representing the in-focus state of the light receiving optical system for the anterior ocular segment of the examinee's eye on the basis of an output of the image pickup element for a specific part of the anterior ocular segment of the examinee's eye; and
an in-focus position determination step of determining an in-focus position of the light receiving optical system based on the focus evaluation value.

10. A control method for an ophthalmic apparatus according to claim 9, wherein the specific part is a pupil edge.

11. A control method for an ophthalmic apparatus according to claim 9, wherein the specific part is an iris pattern.

12. A control method for an ophthalmic apparatus according to claim 9, further comprising an area selection step of selecting an area as the specific part.

13. A control method for an ophthalmic apparatus according to claim 12, wherein in the area selection step, at least one of the position and the size of an area in which the focus evaluation value is acquired is changed dependently on the size of the specific part.

14. A control method for an ophthalmic apparatus according to claim 9, wherein the light receiving optical system includes a focusing member which can be shifted along the direction of the optical axis of the image pickup element, and the in-focus position determination step is a step of changing the position of the focusing member on the optical axis relative to the examinee's eye.

15. A control method for an ophthalmic apparatus according to claim 9, further comprising a main body drive step of driving the light receiving optical system and the image pickup element along the direction of the optical axis of the image pickup element, wherein in the in-focus position determination step, the position of the light receiving optical system on the optical axis is changed by the main body drive step.

16. A recording medium in which a program that causes a computer to execute the steps in the control method according to claim 9 is stored.
